(19) United States
(12) Patent Application Publication
(10) Pub. No.: US 2013/0172868 A1
(43) Pub. Date: Jul. 4, 2013

(54) Systems and methods using sensors that resonate at a frequency equal to a resonance frequency of an ablated tissue

(71) Applicant: Bonfeld, (US)

(72) Inventor: Bonfeld, (US)

(21) Appl. No.: 12/357,501

(22) Filed: Jan. 22, 2009

Related U.S. Application Data
(60) Provisional application No. 61/022,681, filed on Jan. 22, 2008.

(51) Int. Cl. A61B 18/18; A61N 1/37; A61N 1/39

(52) U.S. Cl. CPC A61B 18/18; A61N 1/3937; A61N 1/3706
USPC 606/33; 607/5; 607/27

(57) ABSTRACT

A method is provided of tissue ablation during a tissue ablation procedure. Ablation energy is applied by using a tissue ablation device to create an ablation at a tissue site. An ablation endpoint at the tissue site is detected by using an ablation endpoint device with one or more sensors that are positioned to monitor the ablation. The one or more sensors are selected from at least one of, a piezoelectric and a silicon MEMS sensor. Upon detecting the ablation endpoint, delivery of ablation energy to the tissue site ceases.

### CROSS-REFERENCE TO RELATED APPLICATIONS

This application claims the benefit of U.S. Ser. No. 61/022,681, which application is fully incorporated herein by reference.

### BACKGROUND

1. Field of the Invention

The present invention relates generally to detecting endpoints of ablation procedures, and more particularly to systems and methods that use one or more sensors that resonate at a frequency equal to the resonance frequency of the ablated tissue to determine endpoints of ablation procedures.

2. Related Art

During a medical ablation event, using electromagnetic energy including but not limited to RF, microwave and the like, the treating physician needs to know how far the ablation has proceeded in order to not over ablate.

Imaging methods have been used, without much success, to determine the endpoint of a medical ablation procedure.

In some medical applications, transvenous or intravenous ablation catheters with one or more electrodes are inserted into one or more heart cavities or put in contact with external areas of the heart to administer the ablation treatment to kill selected heart tissue. It is difficult to assess when to terminate the administration of the treatment in a manner that identifies when sufficient tissue has been destroyed to provide a clinically efficacious (transmural) linear ablation lesion. Particularly, “blind” or catheter-based ablation of cardiac tissue (such as to treat atrial fibrillation) can be more effective when patient-specific valid endpoints are used to recognize when a clinically efficacious lesion has been created. In the early ablation experience, acute termination followed by non-inducibility of the arrhythmia were used. Because these endpoints correlated poorly with long-term success, however, other parameters have been developed. Impedance and temperature measurements during the delivery of RF energy and the presence of conduction block after delivery of RF energy are the most common endpoints used in clinical practice.

Accordingly, there is a need for improved endpoint determinations during medical ablation procedures.

### SUMMARY

An object of the present invention is to provide improved endpoint determinations devices, and their uses for medical ablation procedures.

Another object of the present invention, acceleration or vibrations sensor devices are provided that are useful for determining the endpoints of medical ablation procedures.

These and other objects of the present invention are provided in a method of tissue ablation during a tissue ablation procedure. Ablation energy is applied by using a tissue ablation device to create an ablation at a tissue site. An ablation endpoint at the tissue site is detected by using an ablation endpoint device with one or more sensors that are positioned to monitor the ablation. The one or more sensors are selected from at least one of, a piezoelectric and a silicon MEMS sensor. Upon detecting the ablation endpoint, delivery of ablation energy to the tissue site ceases.

### BRIEF DESCRIPTION OF THE DRAWINGS

FIG. 1 is a cross sectional side view of a MEMS pressure sensor with selective encapsulation that can be used in one embodiment of the present invention.

FIG. 2 is a top view of the MEMS device of FIG. 1.

FIG. 3 is a cross sectional side view of a MEMS pressure sensor with selective encapsulation.

FIG. 4 is a top view of the MEMS device of FIG. 3.

FIG. 5 is a perspective view of a MEMS device in accordance with a third embodiment of a MEMS device that can be used with the present invention.

FIG. 6 is a cross sectional view of the MEMS device shown in FIG. 5.

### DETAILED DESCRIPTION

In one embodiment of the present invention an endpoint detection device includes one or more sensors. Suitable sensors include but are not limited to an acceleration or vibration sensor and the like. The sensor can be of either piezoelectric or silicon MEMS technologies to determine an endpoint of an ablation process and can be used for other non-ablation applications.

The endpoint detection device can be used in a variety of therapeutic applications including but not limited to, activity monitoring for implantable defibrillators and pace makers, positioning in an ear canal to measure brain trauma, to monitor tissue ablation progress, provide diagnostic information and therapeutic treatment in a variety of application including but not limited to neurology, and the like. In one embodiment the endpoint detection device can be used for long term implantable catheters.

In one embodiment, the endpoint detection device is used to monitor tissue ablation. The ablation can be performed using an ablation source which is typically electromagnetic. Suitable electromagnetic energy sources include but are not limited to, RF, microwave and the like.

With the present invention, one or more sensors are provided that resonate at a frequency equal to the resonance frequency of the ablated tissue. This resonance frequency is different from the frequency of the non-ablated tissue. The sensor can be coupled to an external detection device. The external device indicates when the sensor is excited to its resonance frequency. The sensor can be coupled to the external detection device by cable, wireless and the like. Achieving a specific resonance frequency is used to determine the endpoint of the tissue ablation procedure.

As the ablation process proceeds tissue radiating from the ablation device is effected by the procedure. When the procedure reaches the tissue where the sensor or sensor array is positioned, the detector notifies the physician with an indication that the procedure should now be discontinued.

In one embodiment, the endpoint detection device has an array of sensors that are mounted to or encapsulated in a biocompatible material, or are manufactured from a biocompatible material. The array of sensors is positioned at an ablation site and can fully or partially surround the ablation site. The array of sensors is tuned to a specific resonance frequency of the ablated tissue. This resonance frequency is different from the resonance frequency of the non-ablated tissue. When the ablation has reached the sensor array and thus the desired endpoint, the array of sensors produces an electrical signal.

In one embodiment, the sensor is a piezoresistive sensor that has a substrate with two opposed surfaces. A dielectric insulative layer is on the a first surface of the substrate. A doped semiconductor layer is on top of the dielectric insulated layer. The semiconductor layer has a high resistivity. The doped semiconductor layer is annealed to one or more regions to lower resistivity of the semiconductor layer and define therein one or more sensor gauges of the annealed semiconductor material. Electrical contacts are adjacent to the annealed semiconductor material and overlay at least a portion of the annealed semiconductor material.

One embodiment of a suitable MEMS sensor that can be used with the present invention includes a housing and a sensor die that can be attached to the housing by an epoxy or silicone adhesive. Wire bonds provide an electrical connection between wire bond pads of the sensor die and a lead frame. A protective dam and an encapsulation gel material can be included as disclosed in U.S. Pat. No. 6,401,545, incorporated herein by reference.

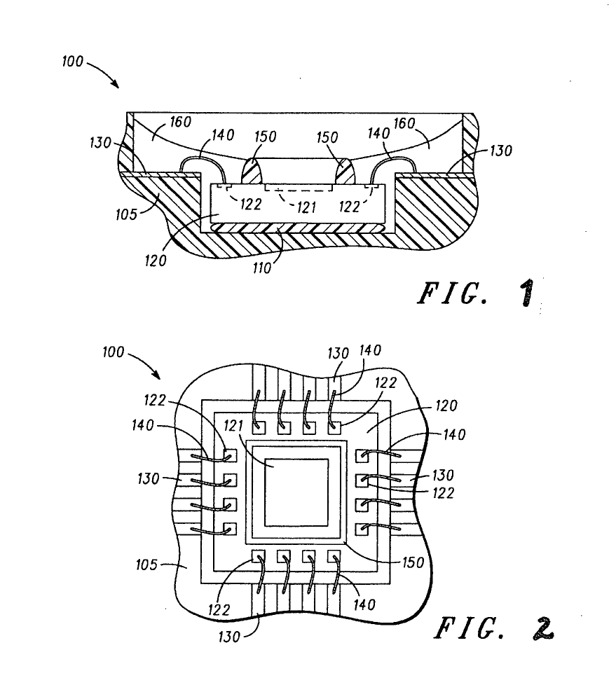

FIG. 1 is a cross-sectional side view of a MEMS sensor 100 with selective encapsulation that can be used in one embodiment of the present invention. MEMS pressure sensor 100 comprises a housing 105 (partially shown) which is typically made of a plastic material. A sensor die 120 is attached to plastic housing 105 by an epoxy or silicone adhesive 110. Wire bonds 140 provide an electrical connection between wire bond pads 122 of sensor die 120 and a lead frame 130. Also shown are a protective dam 150 and an encapsulation gel material 160, which serves as a protectant.

The particulars of the various elements, as well as the technique for fabricating the improved MEMS sensor 100, is as follows. The description of the various embodiments of the present invention is drawn primarily to a MEMS pressure sensor. However, the described embodiments of the present invention of selective encapsulation are applicable to a wide variety of MEMS sensors, including capacitive sensors which sense pressure, chemical, humidity, etc.

The common denominator of these types of MEMS sensors with regard to the various embodiments of the present invention, is a transducer element such as a capacitive diaphragm or membrane which is sensitive to some ambient condition and which, for optimal performance, should be free of encapsulation gel. However, the remainder of the package, other than the transducer element, should be encapsulated for environmental protection.

After a wafer containing numerous MEMS pressure sensor devices is diced into individual dies, each individual pressure sensor die 120 is attached to a housing 105, which is typically made of plastic, by conventional means. Typically an epoxy or silicone adhesive 110 is used to attach pressure sensor die 120 to the base of plastic housing 105.

Subsequent to attaching pressure sensor die 120 to plastic housing 105, wire bonds 140 are connected between die wire bond pads 122 and lead frame 130.

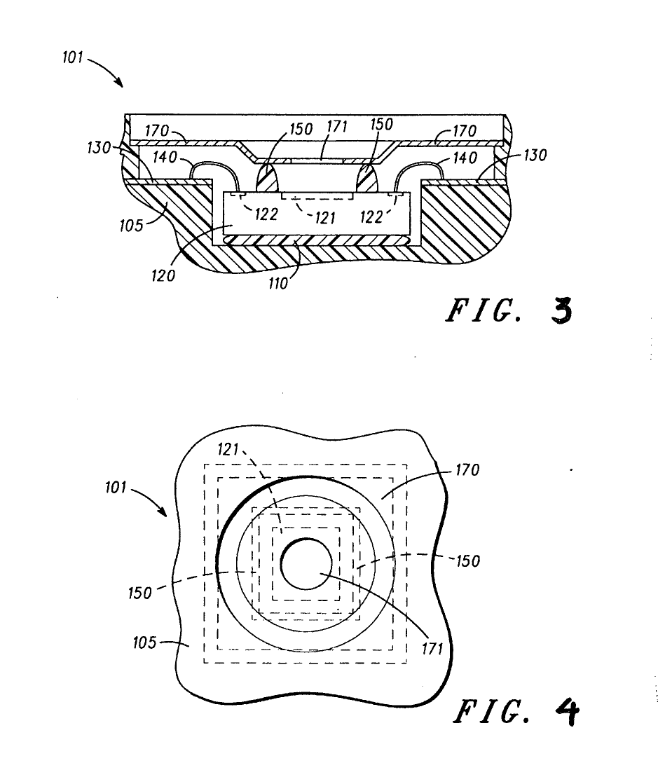

A subsequent step is to construct protective dam 150 between the outer perimeter of a pressure sensor diaphragm 121 and the inner perimeter formed by bond pads 122. Pressure sensor diaphragm 121 is typically located in the center portion of pressure sensor die 120 as shown in FIG. 3. Protective dam 150 is then cured at high temperature.

Following the curing of protective dam 150, MEMS pressure sensor 100 is ready for encapsulation. During this step an encapsulation gel 160 is dispensed into the wire bond cavity region that is located between protective dam 150 and plastic housing 105, thereby covering bond pads 122, portions of lead frame 130 and wire bonds 140. After selective encapsulation is completed, encapsulation gel 160 is cured.

By way of example, protective dam 150 is constructed of a fluorocarbon based material to achieve the best media compatibility, i.e., to protect the integrity of the wire bonds 140 from contamination by foreign matter. However, fluorocarbon type material is also typically the most costly. Other cost effective materials include silicone and fluorosilicone base materials. Typically similar materials are used for both protective dam 150 and encapsulation gel 160.

Protective dam 150 is typically constructed by forming it as a unit using a device such as a dispensing collet. A dispensing collet is a nozzle type device where the design of the output opening of the nozzle corresponds the design of protective dam 150. Thus, for rectangular shaped dams, the dispensing collet would have a rectangular shaped nozzle which would permit the formation of all four walls of the protective dam 150 simultaneously. The current design preferably uses a rectangular shaped protective dam 150 for consistency with the rectangular shaped pressure sensor diaphragm 121. However, other shape diaphragms and protective dams are contemplated including, but not limited to, circular configurations, triangular configuration, pentagonal configurations, or the like.

Alternatively, each of the four walls of protective dam 150 may be formed using a dispensing needle. In the dispensing needle method of protective dam construction, each of the dam walls is formed sequentially, as opposed to the dispensing collet method in which the dam walls are formed simultaneously. The dispensing needle essentially line draws each dam wall. Multiple passes for each wall can be made to control the height and width of protective dam 150.

The minimum height for protective dam 150 is preferably equal to approximately the loop height of wire bonds 140, i.e. the apogee of wire bonds 140 above pressure sensor die 120. The minimum height is driven by the requirement to insure complete encapsulation of wire˜bonds 140. The maximum height of protective dam 150 is that of plastic housing 105. However, in practice the height of protective dam 150 ranges between the apogee of wire bonds 140 and plastic housing 105 as shown in FIG. 2.

For typical applications where the thickness of pressure sensor die 120 is approximately 645 microns (.mu.m), i.e., approximately 25 mils, and the total cavity height of the plastic housing 105 is approximately 135 mils, the nominal height of the protective dam 150 is in the range of 774-1,548 .mu.m, i.e., approximately 30-60 mils. Now referring to FIGS. 3 and 4, MEMS pressure sensor 101 with selective encapsulation 101 in accordance with another embodiment is depicted in which a vent cap 170 serves as a protectant. MEMS pressure sensor 101 includes vent cap 170 covering, sealing or otherwise encapsulating the wire bond cavity region instead using an encapsulation gel to fill the wire bond cavity. Vent cap 170 has a vent aperture 171 in the center which permits pressure sensor diaphragm 121 to receive unmolested ambient pressure. The prior art problem of gel over expansion is avoided by not having to fill the wire bond cavity with encapsulation gel.

Formation MEMS pressure sensor 101 employs similar steps as described for the formation of MEMS pressure sensor 100 including attaching pressure sensor die 120 to plastic housing 105 (partially shown), wire bonds 140 which electrically connect pressure sensor bond pads 122 to lead frame 130, and the construction of protective dam 150.

However, after protective dam 150 has been constructed on a top surface of pressure sensor die 120, vent cap 170 is placed over the device. The outer edges of vent cap 170 mate with plastic housing 105. The lower surface of the center portion of vent cap 170 is pressed down against protective dam 150. Sealing vent cap 170 takes place by curing the device at high temperature. Alternatively, an adhesive material can be used to seal vent cap 170. Also, various combinations of heat curing and adhesive may be employed to seal vent cap 170.

Preferably, vent cap 170 is formed from a plastic material which is compatible with plastic housing 105 and protective dam 150. In alternative embodiments, vent cap 170 may be constructed from metal. However, for a metal embodiment, adequate clearance must be provided between vent cap 170 and wire bonds 140 so as to preclude electrical shorting of wire bonds 140 to vent cap 170. The limitations of the height of protective dam 150 are similar to those described for MEMS sensor 100.

As shown in FIG. 3, vent cap 170 has an offset in the center portion where it contacts protective dam 150. The purpose of the offset is to optimize the height of the protective dam 150 with respect to wire bonds 140 and plastic housing 105. However, alternative embodiments may not need the offset.

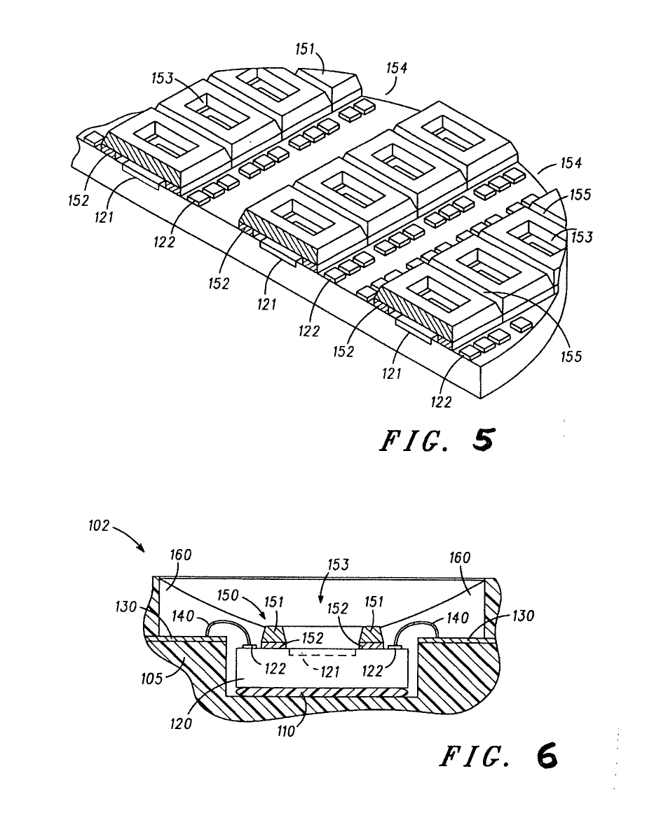

Now referring to FIG. 5, a MEMS pressure sensor 102 with selective encapsulation in accordance with yet another embodiment of the present invention is depicted. In this embodiment, protective dam 150 is formed at the wafer level by bonding a cap wafer 151 to a device wafer 125 by means of a glass frit 152 or other suitable adhesive. A preliminary step in the fabrication of MEMS sensor 102 is to form a plurality of sensor devices on a substrate such as device wafer 125. FIG. 5 illustrates diaphragms 121 and wire bond pads 122 of a typical sensor device.

Independent of the sensor device formation on device wafer 125, a second wafer sometimes referred to as a cap wafer 151 is patterned with a plurality of diaphragm apertures 153, device channels 154 and cut lines 155. A subsequent step is to form a bonding area by depositing a glass frit pattern by screen printing or other means on cap wafer 151. Cap wafer 151 is then aligned and bonded to device wafer 125. The cap/device wafer combination is then heat cured and diced into individual pressure sensor dies 120 having a protective dam 150 attached.

FIG. 6 is a cross sectional view of encapsulated device 102 which further illustrates diaphragm aperture 153. Each of pressure sensor dies 120 is attached to housing 105 as described in previous embodiments. Wire bonding is similarly accomplished by connecting wire bonds 140 between wire bond pads 122 and lead frame 130. A wire bond cavity region is formed between the protective dam 150, i.e., the combination of portions cap wafer 151 and glass frit pattern 152, and housing 105. The wire bond cavity is filled with lo encapsulation gel 160 similar to the previously described embodiments. The limitations of the height of the protective dam 150 are similar to those described with respect to MEMS sensor 100.

In one embodiment, the sensor die includes a transducer element, including but not limited to a capacitive diaphragm or membrane, that is sensitive to some ambient condition and which, for optimal performance, should be free of encapsulation gel material.

In one embodiment, the sensor is a piezoelectric sensor with two metal plates to sandwich a crystal and make a capacitor. External force cause a deformation of the crystal and results in a charge which is a function of the applied force. In its operating region, a greater force results in more surface charge. This charge results in a voltage ^{v=}^{Q}^{₁}^{/L}, where ^{Q}^{₁} is the charge resulting from a force f, and C is the capacitance of the device.

The piezoelectric crystals act as transducers which turn force, or mechanical stress into electrical charge which in turn can be converted into a voltage. Alternatively, if a voltage is applied to the plates, the resultant electric field causes the internal electric dipoles to re-align which cause a deformation of the material.

Although the invention has been particularly shown and described with reference to a preferred embodiment thereof, it will be understood by those skilled in the art that changes in form and detail may be made therein without departing from the spirit and scope of the invention.

What is claimed is:

1. A method of tissue ablation during a tissue ablation procedure, comprising: apply ablation energy by using a tissue ablation device to create an ablation at a tissue site; and
detecting an ablation endpoint at the tissue site by using an ablation endpoint device with one or more sensors that are positioned to monitor the ablation, the one or more sensors being selected from at least one of, a piezoelectric and a silicon MEMS sensor;
detecting the ablation endpoint; and
ceasing delivery of ablation energy to the tissue site.

2. The method of claim 1, wherein the tissue ablation device is an electromagnetic tissue ablation device.

3. The method of claim 1, wherein the one or more sensors resonate at a frequency equal to a resonance frequency of the ablated tissue.

4. The method of claim 3, wherein the resonance frequency is different from a frequency of non-ablated tissue.

5. The method of claim 1, wherein the one or more sensors is coupled to an external detection device.

6. The method of claim 5, wherein the external device indicates when the one or more sensors is excited to its resonance frequency.

7. The method of claim 6, wherein the external device is coupled to the one or more sensors by at least one of, cable and wireless.

8. The method of claim 3, wherein achieving a specific resonance frequency is used to determine the endpoint of the tissue ablation procedure.

9. The method of claim 1, wherein as the ablation procedure proceeds tissue radiating from the ablation device is effected by the ablation.

10. The method of claim 9, wherein the the procedure reaches tissue where the one or more sensors is positioned, the detector notifies the physician with an indication that the procedure should be discontinued.

11. The method of claim 1, In one embodiment, the endpoint detection device has an array of sensors that are mounted to or encapsulated in a biocompatible material.

12. The method of claim 11, wherein the array of sensors is positioned at the tissue site and is at least one of fully and partially surround the tissue site.

13. The method of claim 12, wherein the array of sensors is tuned to a specific resonance frequency of ablated tissue.

14. The method of claim 1, wherein the one or more sensors are piezoresistive sensors with substrates and two opposed surfaces.

15. The method of claim 14, wherein a dielectric insulated layer is on a first surface of a substrate.

16. The method of claim 15, wherein a doped semiconductor layer is on a top of the dielectric insulated layer.

17. The method of claim 16, wherein the doped semiconductor layer has a high resistivity.

18. The method of claim 17, wherein the doped semiconductor layer is annealed to one or more regions to lower resistivity of the semiconductor layer and defines therein one or more sensor gauges of the annealed semiconductor material.

19. The method of claim 18, wherein one or more electrical contacts are adjacent to the annealed semiconductor material and overlay at least a portion of the annealed semiconductor material.

20. A method of activity monitoring implantable defibrillators or pace makers, comprising:
positioning one or more sensors to monitor activity of an implantable defibrillator or pace maker; and
in response to the monitoring taking an action relative to the implantable defibrillator or pace maker.
